(19) United States
(12) Patent Application Publication
(10) Pub. No.: US 2014/0316292 A1
(43) Pub. Date: Oct. 23, 2014

(54) Circulation Monitoring System

(71) Applicant: Semler Scientific, Inc., Portland, OR (US)

(72) Inventors: Bob McRae, Arvada, CO (US); Paul Mannheimer, Danville, CA (US); James McNames, Portland, OR (US); Doug Murphy-Chutorian, Palo Alto, CA (US)

(73) Assignee: Semler Scientific, Inc., Portland, OR (US)

(21) Appl. No.: 13/866,731

(22) Filed: Apr. 19, 2013

(51) Int. Cl. A61B 5/0295; A61B 5/00

(52) U.S. Cl. CPC A61B 5/0295; A61B 5/6826; A61B 5/6829
USPC 600/504

(57) ABSTRACT

A peripheral arterial flow obstruction detection system for providing a predictive diagnosis correlating to the diagnosis peripheral arterial disease. The system includes a host computer and a sensor used to detect and measure a physiological signal from a subject's finger or toe, such as the measurement of a signal using photoplethysmography using a PPG sensor. Sensor data is processed and filtered before being used to calculate a number of time-domain and frequency-domain calculations corresponding to the signal waveform. The calculations are used in a predictive model using a multi-faceted algorithm to provide a predictive diagnosis that is displayed on an indicator such as a monitor.

### RELATED APPLICATIONS

This application relies on concepts drawn from U.S. application Ser. No. 12/001,505 filed on Dec. 11, 2007, titled CIRCULATION MONITORING SYSTEM AND METHOD, now issued as U.S. Pat. No. 7,628,760 on Dec. 8, 2009, the contents and disclosure of which are hereby expressly incorporated by reference in its entirety.

### FIELD OF THE INVENTION

The invention relates generally to the field of medical monitoring. More particularly, the invention relates to circulation monitoring and signal processing to indicate a subject's susceptibility to peripheral arterial disease, which is marked by flow obstruction.

### BACKGROUND OF THE INVENTION

Peripheral artery disease (PAD), as well as related coronary heart disease (CHD) and carotid vascular disease (CVD), are potentially fatal.

In the United States, an estimated 10 million people have PAD. Approximately the same number are deemed undiagnosed due to lack of symptoms and the relative inaccessibility of diagnostic equipment. Disease endpoints for PAD are severe, i.e., disability, limb amputation, and death. It is desirable to enable earlier intervention and avoidance of many of the disease's more severe outcomes by providing easier and more accessible tools to help primary care physicians identify patients with PAD, CAD, CVD, and other co-morbidities in the early stages of the disease.

While PAD is generally associated with lower extremity atherosclerosis, it is associated with an elevated risk of CHD, CVD, heart attack, stroke, and amputation. Approximately 75% of patients having PAD also have CHD or CVD. Risk of stroke is three times higher in patients with PAD than in those without the condition. PAD manifests as stenosis or obstruction of the arteries typically in the lower extremities and is caused by several factors, including atherosclerosis, thrombosis, arterial calcification, diabetes, and homocysteinemia. PAD is a progressive chronic disease characterized by calf pain and disability, specifically claudication, and restricted ambulation due to critical limb ischemia. However, it should be noted that approximately half of all patients with PAD were asymptomatic at the time of their diagnosis.

Current diagnostic methods are typically applied to patients who present with symptoms of claudication or leg pain at rest. A common diagnostic pathway includes use of the Ankle-Brachial Index (ABI) either at rest or post exercise, reactive hyperemia, photoplethysmography (PPG), segmental blood pressure analysis, pulse volume recording, duplex ultrasound, and peripheral angiography.

The ABI is typically the first test deployed and is usually performed in a physician's office or hospital vascular laboratory. The ABI is calculated from observations of systolic blood pressures taken from the brachial artery and at the ankle using sphygmomanometers and Doppler ultrasound. Although the ABI is considered the standard for non-invasive diagnosis of PAD, it is time-consuming, awkward to deploy, subjective, and technique-dependent. To obtain consistent and reliable results, the practitioner must be highly experienced and have specialized training. Further, the ABI is not a useful diagnostic in patients with arterial calcification, a condition commonly encountered in patients at risk for PAD. This fact is because ABI relies on compression of stiff calcified arteries. Such a condition often results in a false negative diagnosis.

Conventional photoplethysmography systems measure the cardio-rhythmic volume of blood in a region of a subject's tissue. Conventional pulse oximeters measure how much oxygen binds to hemoglobin in red blood cells in a region of a subject's tissue. Neither photoplethysmography nor pulse oximietry provide direct correlation to blood flow or circulation quality.

### SUMMARY OF THE INVENTION

The present invention relates generally to sensing micro-vascular perfusion distal to an occlusion and subsequent signal processing. More particularly, it involves a small finger or toe format sensor having integral light generation and sensing components that is operably connected to a host computing platform. The host computer, which includes a processor and memory, implements signal processing algorithms to assess the quality of flow at distal locations of a body extremity.

The detection system includes a housing that is contoured and adapted to fit comfortably over a finger or toe of a test subject. Within the housing is a sensor, which is capable of detecting and measuring a physiological signal. Physiological signals being measured may include, for example, detecting arterial blood flow using a plethysmography sensor. Other physiological signals being measured may include infrared light, organ volume, dermal temperature, dermal impedance, micro-vascular blood velocity, or dermal tension. These signals may be measured a number of different type of sensors, such as a photodiode, a charge coupled device, a pressure cuff, electrodes or strain gauges.

The housing and the sensor are operably coupled to a host computer. The host computer is configured to receive the data as a waveform from the sensor and process it using signal processing techniques. Examples of signal processing techniques include signal artifact detection, normalization, signal filtering, and other time and frequency-domain techniques.

From the sensor signal waveform, a number time-domain feature and frequency-domain feature values that characterize the signal waveform are calculated by the host computer. Examples of the values calculated include a circulation index, harmonic slope, harmonic intercept, systolic rise, spectral signal, spectral noise, and spectral signal-to-noise ratios. Measurements are taken from each limb of or at other various locations on a test subject.

Using the calculated values, the host computer generates a predictive diagnosis using the calculated values as inputs. The predictive diagnosis is calculated using a predictive model equation that is specific to the type of the sensor used and the type of signal gathered. The specific variables and coefficients used in the predictive model equation are generated by performing logistic regression on a sensor data obtained a sample group of measurements made on test subjects with known diagnoses.

### BRIEF DESCRIPTION OF THE DRAWINGS

FIG. 1 shows the computer host connected to a sensor, placed on an index finger of a test subject.

FIG. 2 shows the sensor placed on the second toe of a test subject.

FIG. 3 shows an example a photoplethysmography (PPG) waveform obtained from a patient with low physiological signal amplitude coupled with a high level of low frequency noise.

FIG. 4 shows the corresponding spectral density diagram of the PPG waveform in FIG. 3.

FIG. 5 is a process flow diagram for determining a Pulsatility Index (PI) in accordance with one embodiment of the invention.

FIG. 6 shows an example of a spectral power diagram of a spectral density estimation of a PPG waveform for calculating spectral signal and spectral noise variables.

FIG. 7 shows an example of a spectral power diagram of a spectral density estimation of a PPG waveform for calculating the harmonic decay variables.

FIG. 8 illustrates calculations of systolic rise period variables based on the time-domain features of a PPG waveform.

FIGS. 9A and 9B represent a process flow diagram for a prediction model based on a multi-faceted algorithm in accordance with one embodiment of the invention.

### DETAILED DESCRIPTION OF THE EMBODIMENTS

In one embodiment of the peripheral arterial flow detection system, the system includes a host computer and a sensor for collecting a physiological signal from a test subject or patient. In another embodiment, the host computer may alternatively take the form of a notebook computer, tablet, mobile smartphone, or a remote server.

The host computer is configured to run a software application, which displays data and allows the user to interact with the system. In addition, the system includes a sensor configured to interface with the host computer. The interface may in one embodiment take the form of a hardwired connection through a USB cable.

In another embodiment, the sensor may interface with the host computer through any number of known wireless protocols, such as BlueTooth or IEEE 802.11. Those skilled in the art will recognize that the software application may run on the host computer, within the sensor itself, or on a remote server over the Internet.

In one embodiment, a physiological signal is collected from a test subject or patient through the sensor. In one embodiment, the physiological signal is a photoplethysmography (PPG) waveform collected using a PPG sensor. It will be understood that while a PPG waveform is in the form of a pulse waveform, use of different types of sensors to collect other physiological signals will not necessarily result in a pulse waveform.

Data from the sensor is transferred to the host computer for processing. The host computer displays the data collected from the sensor. The host computer may also calculate a number of values based on the PPG waveform. Using the values characteristic of the PPG waveform, the host computer is configured, such as through a software application, to calculate a predictive diagnosis and display the results on a monitor or display.

In addition, one of skill in the art will appreciate that a variety of different sensor types may be used to collect a variety of physiological signals, including not limited to photoplethysmography (PPG), Laser Doppler Velocimetry (LDV), infrared charged coupled device (CCD), a pressure cuff configured with a pressure transducer, dermal impedance electrodes, and dermal strain gauges.

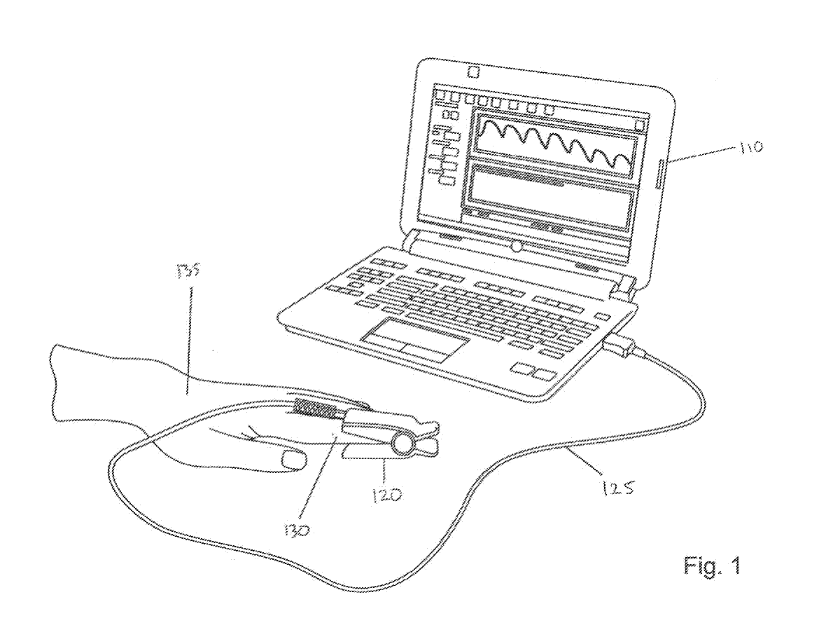

With reference to FIG. 1, in one embodiment a host computer 110 is connected to a housing 120 by a USB cable 125. The host computer 110 is running a software application that displays data collected from the housing 120.

The housing is contoured to receive a portion of a peripheral limb, such as a finger or a toe. Located within the housing 120 is a sensor for detecting and measuring a physiological signal. In one embodiment, the sensor is a PPG sensor that detects and measures arterial flow and generates a photoplethysmographic signal in the form of a pulse waveform.

It will be understood, as previously described, that the PPG sensor may be substituted with other types of sensors for collecting different types of physiological signals. It will also be understood that the interface, shown as USB cable 125, may be substituted with other wired or wireless data interfaces without loss of functionality.

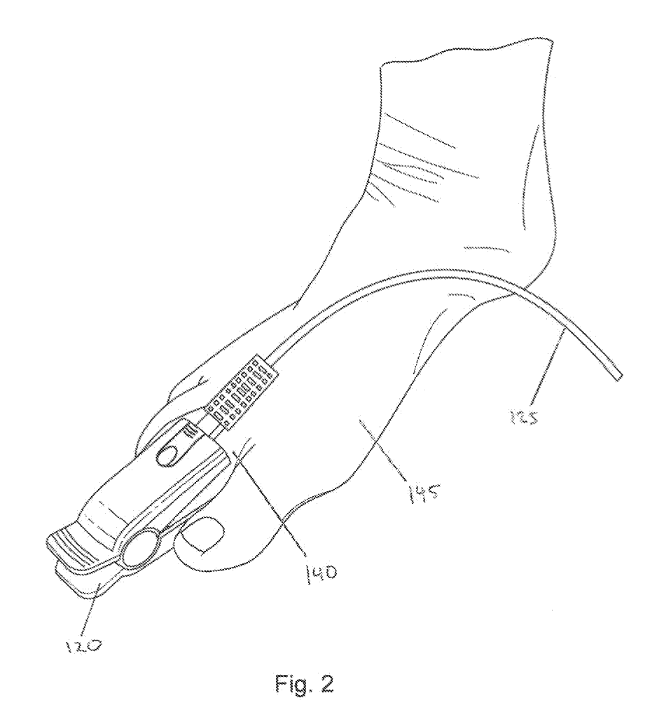

Although in FIG. 1 the housing 120 is shown as attached to the index finger 130 of the left hand 135, the housing 120 may be attached to other bodily appendages. For example, with reference to FIG. 2, the housing 120 may be attached to the second toe 140 of a test subject's right foot 145. As further described herein, it may be desirable to take sensor readings from other appendages, organs, or locations on the body, such as fingers, toes, feet, hands, legs, ears, forehead, supraorbital, and epidermis.

In one embodiment, a netbook computer may be used as the host computer. For example, the host computer may be configured to run a Microsoft® Windows® based operating system or the like. Typically, such a computer will include a central processor, such as the Atom N455 CPU and include approximately 1 GB of random access memory (RAM). In addition, such a computer will typically include means to connect to external devices, including a network card, a BlueTooth card, and universal serial bus (USB) ports. Additionally, such a PC may include a small LCD display.

Alternative computing hosts may include a tablet, running Apple's iOS, an Android tablet, or Windows tablet. Smaller host computers facilitate portability in that they are easier for healthcare practitioners to carry between patients.

In one embodiment, the software application may be written in C#. Alternatively, several different languages may be suitable, including: Java, MatLab, C, C++, AJAX, Javascript, Perl, Ruby, Python, VB, and the like.

It will be understood that depending on what physiological signal is being collected, different sensors can be used. In one embodiment, the sensor is a plethysmography sensor for measuring the relative blood flow through a finger or a toe. It will be understood that plethysmography sensors refer to a class of sensors for plethysmography, including for example photoplethysmography (PPG) sensors, inductive plethsymography sensors, strain gauge plethysmography sensors, and volume plethysmography sensors. In one embodiment, the PPG sensor includes an infrared light emitting diode (IR LED), operating at 940 nm paired with a photodiode sensitive to a similar wavelength. It will be understood that other wavelengths may also be used, such as 630 nm and 535 nm. The PPG sensor may also include a microcontroller unit (MCU), which may be programmed to perform many of the operations as described in this specification. In another embodiment, the MCU of the PPG sensor may serve the function of the host computer itself. Examples of suitable MCUs are manufactured by Texas Instruments, such as the MSP430 series MCUs. Other components may also be included in the PPG sensor, including an analog to digital converter (ADC) such as model LTC2451 from Linear Technologies or a digital to analog converter (DAC) such as model LTC1669 from Linear Technologies.

The previously described PPG sensor may be used to provide a data signal such as voltage output, current, or ADC bit counts. This data signal may then be further processed by the host computer.

Although an embodiment has been described using a PPG sensor, it is understood by those of skill in the art that other sensor types may be used for obtaining vascular physiological signals. For example, other researchers have demonstrated the utility of various sensor types for the purpose of monitoring vascular perfusion, such as described in the following references, the disclosures of which are hereby expressly incorporated by reference in their entirety:

Laser Doppler Velocimetry: Fischer M, et al, “Simultaneous measurement of digital artery and skin perfusion pressure by the laser Doppler technique in healthy controls and patients with peripheral arterial occlusive disease.”, Eur J Vasc Endovasc Surg. August 1995; 10(2):231-6. Such a system uses a laser to illuminate a local region of epidermis and then measures the back-scattered laser light. This back-scattered light undergoes a frequency shift proportional to its velocity. Thus, the signal of interest may be generated and recorded in terms of raw laser light, voltage, current, or ADC counts which are related to the phase shift.

Infrared CCD: Karel J. Zuzak, et al, “Visible spectroscopic imaging studies of normal and ischemic dermal tissue.” Proc. SPIE 3918, Biomedical Spectroscopy: Vibrational Spectroscopy and Other Novel Techniques, 17 (May 8, 2000). In the case of an infrared CCD, the signal may be generated and recorded in the form of wavelength, voltages, currents, or ADC counts.

Pressure cuff and transducer: Biomedix, Inc. (http://www.biomedix.com/products/PADnet_plus.asp). Such a pressure transducer may produce a current or voltage related to pressure or pressure changes. These raw signals may be further digitized to allow for further processing.

Impedance Plethysmography: Jane C Golden and Daniel S Miles, “Assessment of Peripheral Hemodynamics Using Impedance Plethysmography”, PHYS THER. 1986; 66:1544-1547. As the name implies, changes in skin impedance are measured with dermal electrodes. The output signal is typically an electrical current, which may be digitized through an ADC and relayed to the host computer.

Strain Gauge Plethysmography: Myers, Kenneth, “The Investigation of Peripheral Arterial Disease By Strain Gauge Plethysmography”, ANGIOLOGY July 1964 15: 293-304. This modality is accomplished via a strain gauge affixed to the epidermis, typically in an circumferential manner around the organ being measured. Thus, arterial perfusion results in volumetric changes in the organ, which tends to change the resistance of the strain gauge. This analog voltage may be digitized and relayed to the host computer for processing.

In each of the above mentioned technological modalities, arterial perfusion is sensed and relayed to the host computer as a signal for further processing.

### Multi-Faceted Algorithm

The operation of the multi-faceted algorithm (MFA) will be now be described. Although the description is provided in the context of PPG waveforms collected from a PPG sensor from the different limbs of a test subject or patient, it will be understood that the methodology of the MFA can be used to analyze and derive predictive diagnoses using physiological signal data collected at various bodily locations using other types of sensors such as those previously described.

Signal traces obtained from a sensor such as photoplethysmography (PPG) can be analyzed for morphological features. The morphological features in a PPG waveform are indicative of a limb's disease state. Morphological features of a PPG waveform may include time-domain features such as the systolic rise and the normalized signal. Morphological features of a PPG waveform may also include frequency-domain features such as the harmonic slope, harmonic intercept, spectral signal, and spectral signal-to-noise ratios. A variety of indices can also be calculated from the PPG waveform, such as a Circulation Index (CI) as described in U.S. Pat. No. 7,628,760 (the disclosure of which is incorporated by reference in its entirety), and a Pulsatility Index (PI) as described herein. Further calculations, as described herein, can be computed from each of these features. The results of those calculations, which characterize the PPG waveform, may be used as input components in a MFA to predict the probability of arterial flow obstruction.

The MFA takes into account not only the spectral flatness of the PPG waveform, but other frequency and time-domain features of the PPG waveform as well. Calculations generated from the morphological features of the PPG waveform are used as inputs into the MFA, which can be used to more reliably predict the probability of flow obstruction than CI or PI alone.

Example of the types of calculations that can be generated based on the morphological features of the PPG waveform are described below.

### Per Limb/Digit Measurements and Calculations

Data is may be collected through the sensor from each limb or digit. Data is relayed from the sensor and analyzed either at the end of a measurement or in real-time. In one embodiment, blood volume measurements are sampled at a rate of approximately 50 times per second. In one embodiment, the measurement of a digit may be of a finite duration, such as 15 seconds, or may be a continuous measurement with a user-determined termination.

Optionally, in real-time, on a periodic basis (e.g., once per second), or at the conclusion of a digit measurement, the measurement is assessed for the presence of a signal artifact. Various methods of artifact detection may be used. Such methods include: bispectrum/bicoherence analysis, DC discontinuity assessment, kurtosis, and skew. In one embodiment, the Sample Kurtosis method is used. The Sample Kurtosis method is represented in Equation 1:

$${g}_{2} = \frac{{m}_{4}}{{m}_{2}^{2}} - 3 = \frac{\frac{1}{n} {\sum }_{i = 1}^{n} {( {x}_{i} - \overline{x} )}^{4}}{{( \frac{1}{n} {\sum }_{i = 1}^{n} {( {x}_{i} - \overline{x} )}^{2} )}^{2}} - 3$$

Measurements that do not have a signal artifact are marked with low kurtosis measure, such as less than 1.0. In one embodiment, a kurtosis threshold of 1.0 is able to achieve a sensitivity of 97% and specificity of 97% in the detection of clinically relevant artifacts.

As the sensor data represents reflected light as opposed to absorbed light, subtraction is a simple means of transforming the signal data in a manner that represents absorbed light, which is directly related to volumetric changes in the imaged arteriole bed. In addition, further time-domain calculations rely on the proper temporal orientation of the PPG waveform. In addition, a normalization step may be performed which yields a signal that is a direct measure of blood volume changes. It is computationally efficient and convenient to subtract and normalize the signal with a single mathematical equation such as in the following form (Equation 2):

$$normData = 1 - \frac{rawData}{initialMedian} , \qquad ( 2 )$$

where, rawData is the data from the sensor and initialMedian is the median of the initial raw data, typically three points. The initial median is used to normalize rather than the initial point to allow for initial transient conditions or other anomalies. It should be recognize that other normalization techniques may be used such as initial average (of the first n-points), moving average of m-points (e.g., 1-second), overall average, or a fixed nominal value, such as the data associated with a nominal level (e.g., 32,768 ADC points from a 16-bit ADC).

In addition, other subtraction techniques may be used such as, initial median (of the first n-points), initial average (of the first m-points), moving average (e.g., 1-second), overall average, fixed nominal (e.g., 32,768 ADC points from a 16-bit ADC) or fixed maximum (e.g., 65,536 ADC points from a 16-bit ADC).

Normalizing the signal by an amount representative of the average signal level (DC) yields a peak-to-trough (AC/DC component), which may be clinically relevant. It should be appreciated that doubling the amount of average light from the illumination source would be accompanied by a doubling of the raw AC component if it was not otherwise normalized by the DC value, demonstrating the need for the application of a dynamic normalization technique such as that provided by the initial median.

After the normalization and subtraction, a normalized spectral density of the signal is estimated using a modified version of the Welch's Method. In particular, each spectral density is normalized by dividing each frequency's power by the maximum for that spectral density estimation. This technique reduces the effects of noise, which tend to overwhelm the physiologic signal in absolute power, but due to the transient nature are mitigated by the averaging associated with the Welch Method and the addition of normalization to the method.

For example, assume a physiologic signal occurs at 1 Hz and 5,000 power units and presents relatively consistently through each Fourier Fast Transform (FFT) of the Welch Method. However, if noise presents in only a few of the many FFT's with a power of 10,000 units at a frequency of 0.5 Hz, normalizing transforms that power to 1.0 unit. In addition, noise may only be present in a fraction of the FFT's used in the Welch Method. Due to the noise mitigating nature, normalizing the spectral density allows for a more reliable estimate of the Fundamental Frequency (F0).

Several methods exist for estimating F0, including: prominence, simple maxima, Harmonic Product Spectrum (HPS) and variants thereof. Strong signals which are devoid of noise allow for simple techniques such as simply selecting the maximum power associated with a spectral density. Such selection may be further enhanced and computational efficiencies gained by narrowing the frequency range to, say, 0.6 to 2.5 Hz.

When physiologic signals become confounded with noise, more sophisticated techniques become necessary to better estimate F0. The HPS is one such method, which involves essentially a product of the powers of a given frequency and its associated harmonics. For example, the power of 1.2 Hz is multiplied by the power of 2.4 Hz, 3.6 Hz, 4.8 Hz and so on and so forth. A variant of this method includes sums instead of products.

Another method, which has shown particular utility involves the use of the prominence, or relative height, of the spectral peak as compared to surrounding points. For example, the prominence of 1.2 Hz may be calculated as the ratio of power at 1.2 Hz to the average of powers at 0.8 Hz and 1.6 Hz. Prominence shows particular advantage in spectra which include low-frequency noise where the power is high at low frequencies, but decays as frequency increases. In such a case, the HPS does not differentiate a local peak as well as the prominence method. The width of adjacent comparison (spectral “distance” from the center frequency) is an important parameter in the prominence method.

One choice for a denominator comparison is ½ F0, which should theoretically correspond to the troughs to F0's peak. Empirical testing with noisy clinical data demonstrated that a width 35% was optimal. For example if the presumed F0 was 1.2 Hz, the adjacent comparison frequencies were 0.78 Hz and 1.62 Hz. Equation 3 below shows the calculation of prominence:

$$π ( f ) = \frac{2 P ( f )}{P ( f - λ ) + P ( f + λ )} , \qquad ( 3 )$$

where P(f) is the power at a given frequency and a, is the offset width. Selection of F0 is based on the associated frequency with maximum prominence.

It should be understood that combinations of prominence and HPS are to be considered within the scope of this invention.

Once an estimate of F0 has been made, the signal may then be filtered to remove low-frequency components with minimal alteration of the signal of interest. That is, by knowing the F0, one may then perform a high-pass filter with a corner frequency that minimally attenuates the F0, but maximally attenuates frequencies below F0.

Performing a high-pass filter essentially de-trends the signal and provides for more reliable time and frequency-domain processing on the signal of interest. Several different methods may be used for high-pass filtering in both the time and frequency-domains, including: Butterworth, Chebyshev, moving average, and convolution.

For example, a 4^{th} order, bidirectional Butterworth filter provides for a sharp attenuation, zero phase shift, and is reasonably computationally efficient and stable. In addition, it provides a flat frequency response in the band-pass range, which is particularly important for further frequency-domain processing which relies on a relatively unadulterated signal. Selecting a corner frequency of approximately 40% of F0 tends to limit attenuation of F0 to around 5%, but effectively attenuates components due to such things as: involuntary movement, ambient light changes, and respiration.

As described in U.S. Pat. No. 7,628,760, the Circulation Index (CI) is a measure of how strong the quasi-periodic component of the signal, which is essentially the compliment of the Spectral Flatness Measure (SFM). Like the SFM, the CI is on a scale of 0 to 1, though it is may sometimes be expressed as a percentage (e.g., 0% to 100%). Although SFM has been used in speech processing and other applications, SFM has not been used in cardiovascular signal applications except in limited circumstances such as those described in U.S. Pat. No. 7,628,760.

Calculation of CI may be improved by removing the low-frequency components of the signal via a variety of detrending methods including: high-pass filtering, linear regression, polynomial fitting, cubic spline fitting, or smoothness priors. Removal of the low-frequency components which are typically associated with physiologic processes and/or environmental conditions not related to volumetric changes in arteriole circulation. That is, respiration, involuntary movements, and ambient light changes may be essentially removed from the signal as a preprocessing step and yield a more clinically significant estimate of the CI.

Although the Spectral Flatness Measure (SFM) is normally defined over the entire frequency range of the PSD, it can also be applied to any band of frequencies. In particular, one embodiment includes the frequency range of 0.6 Hz to 8.0 Hz, which captures the fundamental frequency and the corresponding three harmonics of most patients.

An alternative embodiment uses a Pulsatility Index (PI) instead of or in addition to the CI. As described previously and in U.S. Pat. No. 7,628,760, the CI is correlated with and can be used as a standalone metric to predict the probability of a positive PAD diagnosis.

In some patients, the spectral flatness of the PPG waveform alone may not provide a reliable prediction of a PAD diagnosis. In patients with proximal flow obstruction the PPG waveform may show low physiological signal amplitudes coupled with a high level of low frequency noise. Patient respirations, arrhythmia, low-frequency blood flow changes, or other ambient light and motion artifacts may also affect the spectral flatness of the PPG waveform.

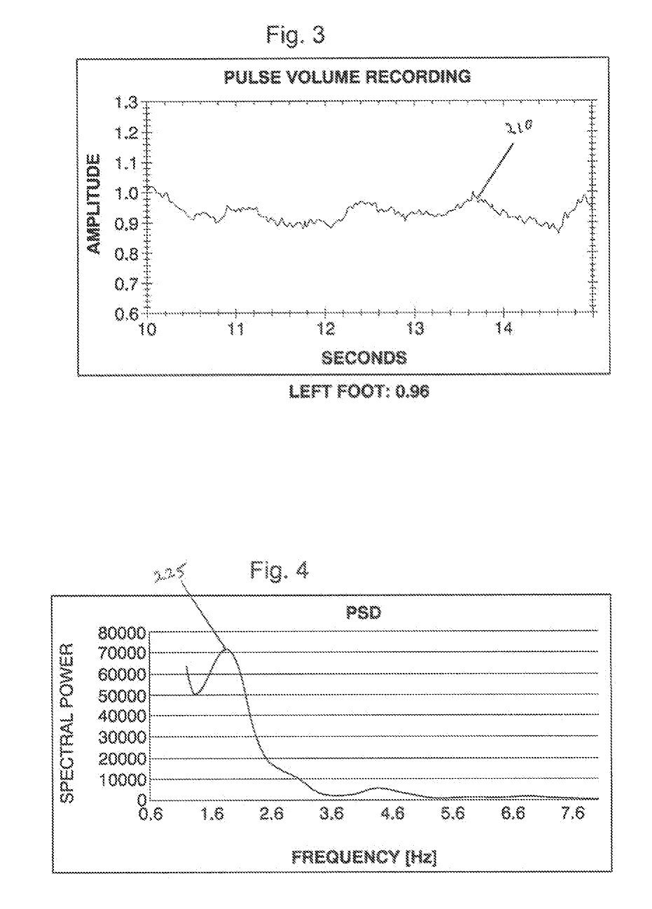

An example of such a PPG waveform taken from the left foot of a test subject is shown in FIG. 3. The PPG waveform 210 is plotted as amplitude over time. As can be observed, the PPG waveform 210 displays a low physiological signal amplitude that is coupled with a high level of low frequency noise. This is more evidence when viewing the corresponding power spectral density diagram for the PPG waveform 210.

As can be seen by the corresponding spectral density diagram in FIG. 4, the waveform is characterized by the prominence of a low frequency peaks 225. This low frequency power shows the presence of a high level of noise. In these types of patients, predictions calculated from the spectral flatness measure alone using methods such as the Circulation Index (CI) described in U.S. Pat. No. 7,628,760 may provide distorted results. This is because the low frequency noise results in a low spectral flatness measure, and as a result an abnormally high CI.

An alternate algorithm may be used in order to overcome such limitations of the spectral flatness based CI as a sole predictor of flow obstruction. This alternate algorithm, referred to as the Pulsatility Index (PI), will now be described.

The Pulsatility Index (PI) addresses this issue by considering the spectral power at the fundamental frequency and its harmonics, compared to the power in the troughs between to marginalize non-physiologic signal noise.

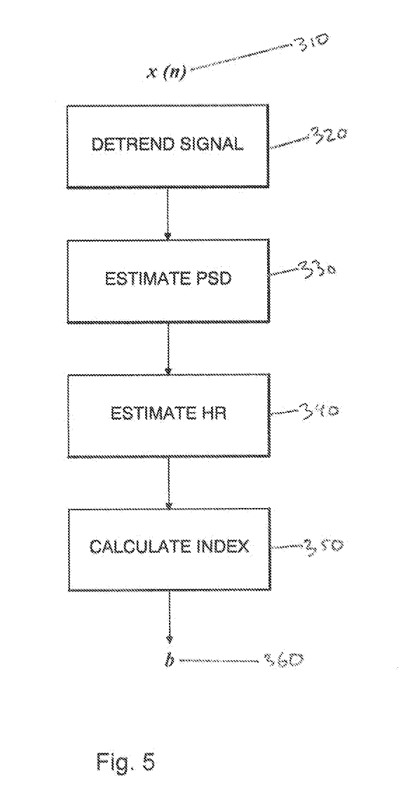

FIG. 5 shows the main process steps of one such algorithm for determining a Pulsatility Index. Beginning with a signal x(n) shown as 310, the first step in determining the Pulsatility Index is to detrend the signal as shown in step 320. This can be accomplished by estimating the trend of the waveform and removing it. The trend may be estimated using any number of methods, including but not limited to: smoothness prior method, linear regression, polynomial fitting, use of a cubic spline, or application of a high-pass or low-pass filter. In one embodiment, the trend of the waveform is estimated using a finite impulse response (FIR) low-pass filter to estimate the trend, as shown in Equation 4:

$${x}_{t} ( n ) = {\sum }_{k = - M}^{M} {h}_{M} ( n - k ) x ( k ) , \qquad ( 4 )$$

where M is the length of the impulse response h(n), n is the discrete-time index, x(k) is the input signal, and xₜ(n) is the output. In one embodiment, the impulse response have equal weightings. In another embodiment, an Epanechnikov kernel is used as the impulse response.

$${h}_{M} ( n ) = \{ \begin{matrix}1 - {( \frac{n}{M} )}^{2} & n < M \\ 0 & n \geq M\end{matrix} , \qquad ( 5 )$$

The width of the kernel is specified in terms of the cutoff frequency. In one embodiment, the algorithm uses a cutoff frequency of 0.5 Hz. At 0.5 Hz, the cutoff frequency is low enough to avoid inclusion of the cardiac cycle in the estimate of the trend, but high enough to include the additive effects of respiration and other elements that affect low frequency drift. Other embodiments may use a dynamic corner frequency based on an estimate of F0. A Fast Fourier Transform (FFT) algorithm can be used to reduce the computational cost of the algorithm. In one implementation, the first and last portions (e.g., 0.5 seconds) are truncated to eliminate the edge effects of the filter.

The detrended signal is then calculated simply as the subtraction of the low-pass signal (the trend) from the original signal:

x_{d}(n)=x(n)−xₜ(n) (6),

After the signal is detrended, the spectral density of the signal is estimated as shown in step 330. Any number of methods may be used to estimate the spectral density, including, but not limited to: Power Spectral Density (PSD), Welch's Method, and Burg's Method. In one embodiment, the spectral density estimation is the Power Spectral Density (PSD). The PSD can be estimated using parametric or nonparametric methods. In one implementation, the PSD is estimated using a Blackman-Tukey spectral estimation. The autocorrelation of the signal is estimated using the standard, biased estimator, as shown in Equation 7:

$${\hat{r}}_{x} ( 𝓁 ) = \frac{1}{N} {\sum }_{n = 0}^{N - \vert 𝓁 \vert - 1} {x}_{d} ( n + \vert 𝓁 \vert ) {x}_{d} ( n ) , \qquad ( 7 )$$

where N is the length of the detrended segment, the segment is indexed from 0 to N-1, and f is an index indicating the delay at which the estimate is calculated.

The autocorrelation is then multiplied by a window wₐ(l) and the discrete time Fourier transform of the windowed autocorrelation function is calculated. Any number of windowing techniques may be used, including, but not limited to: Blackman, Hamming, Hanning, etc. In one embodiment, a Blackman is used, as shown in Equation 8:

$${\hat{R}}_{x} ( ω ) = {\sum }_{𝓁 = - ( L - 1 )}^{L - 1} {\hat{r}}_{x} ( 𝓁 ) {ω}_{a} ( 𝓁 ) {e}^{- j ω 𝓁} , \qquad ( 8 )$$

where ω is an index of frequency. A window should be chosen so that there is sufficient duration to estimate peaks in the PSD due to the cardiac cycle, but also provides sufficient smoothing to significantly reduce the variance of the estimate and eliminate spurious peaks. In one implementation, the Blackman autocorrelation window has a duration of 8 seconds.

After the PSD has been estimated, the heart rate can be estimated as shown in step 340 by searching over a range of possible heart rate frequencies for the heart rate that optimizes a criterion. In one embodiment, searches are made over the frequency range of 0.7 to 2.5 Hz, which corresponds to 42 to 150 beats per minute. The criterion is based on the sum of the powers at the harmonics of the candidate heart rate relative to the powers of the PSD in the troughs between the harmonics. This method is a variant of the prominence method, as previously described. The power at the harmonics is calculated as the value of the PSD at integer multiples of the peaks, as shown in Equation 9:

$${p}_{p} ( ω ) = {\sum }_{k = 1}^{{N}_{h}} {\hat{R}}_{x} ( k ω ) , \qquad ( 9 )$$

where Nₕ is the number of harmonics used. One embodiment uses the fundamental (F0) and three harmonics (F1, F2, F3). The power of the harmonic signal could be estimated with other methods, such as calculating the area of each peak with close, surrounding neighboring frequencies.

The power of the troughs is calculated as the value of the PSD at frequencies in between the harmonics. In one implementation, three frequencies in between each pair of harmonics are used, specifically the midpoint (50% inter-peak point), the 40% inter-peak point, and the 60% inter-peak point, as shown in Equation 10:

$${p}_{t} ( ω ) = {\sum }_{k = 1}^{{N}_{h}} {\hat{R}}_{x} ( ( k + 0.4 ) ω ) + {\hat{R}}_{x} ( ( k + 0.5 ) ω ) + {\hat{R}}_{x} ( ( k + 0.6 ) ω ) , \qquad ( 10 )$$

The criterion for determining the heart rate is calculated as the ratio of the sum of the PSD power in the troughs divided by the sum of the PSD power at the peaks as shown below in Equation 11.

$$ρ ( ω ) = \frac{{p}_{t} ( ω )}{{p}_{p} ( ω )} , \qquad ( 11 )$$

The candidate heart rate with the smallest ratio is chosen as the estimated heart rate, as shown in Equation 12:

ωₕᵣ=argmin ρ(ω) (12),

where function argmin refers to the index associated with the smallest value of p(w).

After the heart rate has been estimated, the Pulsatility Index can be calculated in step 350 as shown in Equation 13:

$$b = \{ \begin{matrix}1 - ρ ( {ω}_{hr} ) & ρ ( {ω}_{hr} ) \leq 1 \\ 0 & ρ ( {ω}_{hr} ) > 1\end{matrix} , \qquad ( 13 )$$

where ρ(ωₕᵣ) is the ratio described in the earlier stage evaluated at the estimated heart rate. The Pulsatility Index is represented by the reference character b 360 in FIG. 5.

Small ratios corresponding to high power at the peak frequencies result in large estimates of pulsatility. Large ratios that correspond to more power in the troughs and less power at the peaks return results that correspond to small estimates of pulsatility.

Returning specifically to the MFA, after low-frequency components have been removed through de-trending, a traditional spectral density estimation may be performed which is not dominated by low-frequency noise. Again, various methods may be used as previously discussed, including the traditional Welch Method. This spectral density estimation may be particularly useful for a more accurate estimation of F0. Typically, the peak power between a certain frequency range such as 0.6 Hz to 2.5 Hz may correspond to F0. Again, various methods of estimating F0 may be used. In one embodiment, the prominence method is used.

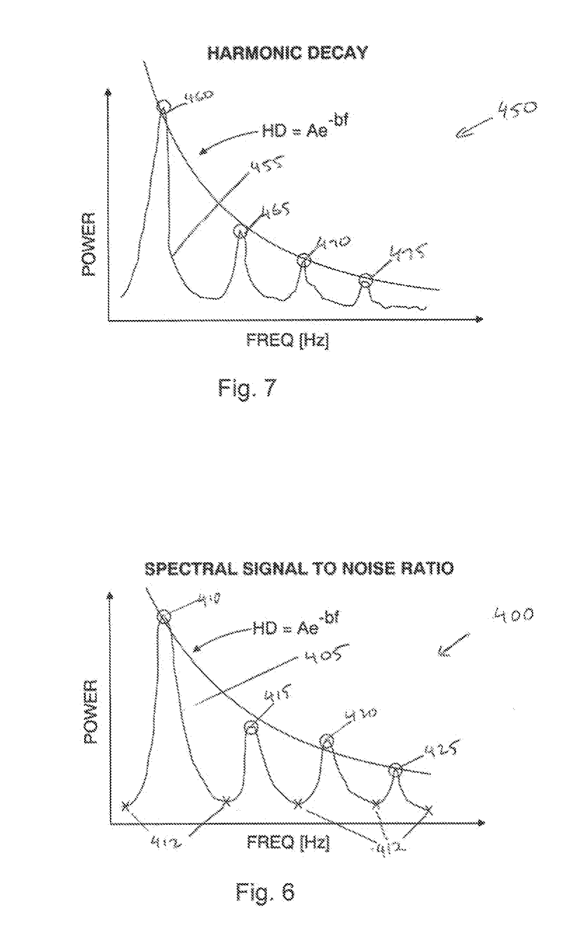

The spectral density estimation provides the basis for the calculation of such variables as spectral signal, spectral noise, and spectral signal-to-noise ratio (SNR). FIG. 6 shows a spectral power diagram 400 of a spectral density estimation of a PPG waveform 405. From the spectral power diagram 400, the fundamental frequency 410 (F0), as well as the first harmonic 415 (F1), second harmonic 420 (F2), and third harmonic 425 (F3) can be identified from the estimated spectral density of a PPG waveform 405.

The spectral signal (“spec.sig”) and the spectral noise (“spec.noise”) can be calculated from:

$$\begin{matrix}spec . sig = {\sum }_{i = 0}^{N} {P}_{i} , & ( 14 ) \\ spec . noise = ( \frac{N + 1}{N + 2} ) {\sum }_{j = 0}^{N + 1} {P}_{j} , & ( 15 )\end{matrix}$$

where Pᵢ is the power at F0, F1, F2, . . . Fn, and Pⱼ is the power at F0/2, 3F0/2, 5F0/2, . . . (N+1)F0/2, which generally corresponds to the “x” marks 412 in FIG. 6.

The Spectral SNR (“spec.SNR”) is calculated as shown in Equation 16 below. It should be recognized that the Spectral SNR shares many similarities with the PI as previously described.

$$spec . SNR = \frac{spec . sig}{spec . noise} , \qquad ( 16 )$$

With reference to FIG. 7, another example of a spectral power diagram 450 of a spectral density estimation of a PPG waveform 455 is shown. Again, the fundamental frequency 460 (F0), as well as the first harmonic 465 (F1), second harmonic 470 (F2), and third harmonic 475 (F3) can be identified from the estimated spectral density of a PPG waveform 455.

Having identified the fundamental frequency and harmonic frequencies, the Harmonic Decay (HD) can be defined using Equation 17:

HD=Ae^{-bf} (17),

where A is the harmonic intercept, b is the harmonic slope, and f is either the harmonic number (0, 1, 2, 3, etc) or the actual corresponding frequency (1.2, 2.4, 3.6, 4.8 Hz, etc.). Either the harmonic number or the actual frequency may be used in the calculation of the Harmonic Decay variables. In some instances, the harmonic number actually provides for a more statistically significant variable than the absolute frequency.

The harmonic slope (“harm.slope”) can be calculated using a standard least squares approach or the LINEST and INDEX functions used in the commercially available software package Microsoft® Excel® using the formula below:

$$harm . slope = INDEX ( LINEST ( ln ( \frac{{P}_{Fi}}{{P}_{F 0}} ) ) , 1 ) \\ i = 0 , 1 , 2 , 3 , … n , \qquad ( 18 )$$

where P_{F0} is the power of the fundamental frequency, and P_{Fi} is the power of the ith harmonic of F0, the first harmonic (P_{F1}), the second harmonic (P_{F2}), etc. While any number of harmonics may be used, sufficient clinical utility may typically be achieved using up to the third harmonic (i=3). The function above shows the harmonic power being normalized with the power of F0 (e.g., P_{Fi}/P_{F0}). An alternative embodiment excludes this normalization.

Similarly, the harmonic intercept (“harm.int”) can be calculated using the formula below:

$$harm . int = INDEX ( LINEST ( ln ( \frac{{P}_{Fi}}{{P}_{F 0}} ) ) , 2 ) \\ i = 0 , 1 , 2 , 3 , … n , \qquad ( 19 )$$

Again, a typical least squares function may be used in place of the above Microsoft® Excel® function. As well, the harmonic intercept may or may not be normalized with the power of F0.

In addition to accurate Harmonic Decay variables, the de-trended signal also provides the basis of an accurate estimate of F0, which is critical in further time and frequency-domain calculations. Again, several methods may be used to estimate F0 as previously described. One implementation uses the prominence method, as described in Equation 3.

Once a reliable F0 has been estimated, the signal may be further filtered to remove high-frequency components, which lie beyond the signal of interest. Again, several methods may be used to remove the high frequency components in both the time and frequency-domains, including: Butterworth, Chebyshev, moving average, and convolution. One implementation, for example, uses a 4^{th} order, bidirectional Butterworth low-pass filter with a corner frequency of 4.5 times F0. For example, if F0=1.2 Hz the corner frequency would be set at 5.4 Hz. Selection of this corner frequency provides to an unaltered signal between F0 and the “trough” following the third harmonic. In general, selection of the corner frequency may be based on the following equation:

F_{c}=(HN+1+0.5)×F0 (20),

where HN is the harmonic number (e.g., 3).

Attenuation of the higher frequency components provides for a signal which includes only the frequency components of interest. Removal of the high-frequency noise, for example, provides a more reliable means of peak and trough detection, or simply “peak detection.”

In addition to the frequency-domain features, a PPG waveform will have a number of time-domain features.

Robust peak detection is critical for a reliable calculation of the Systolic Rise (SR) variable. While a noisy signal benefits from aggressive filtering, the filter specifications should be selected with care to avoid significantly altering the shape of the signal. Hence, selection of the frequency range from 0.40 to 4.5 times F0 provides for a reasonably filtered signal allowing for robust peak detection without distorting the signal of interest.

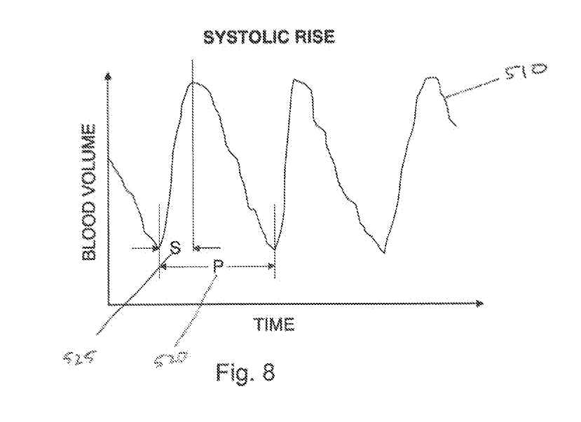

The SR feature of the PPG waveform will be discussed with reference to FIG. 8. The SR of a waveform 510 may be expressed as a percentage of the period 520 (P) and is calculated as:

$$SR = \frac{S}{P} , \qquad ( 21 )$$

where S is the time 525 it takes the waveform to rise from trough to peak and P is the period of the signal. Alternatively, SR may be expressed in absolute terms.

Systolic rise is correlated with PAD in the measured limb, with diseased limbs having longer SR periods. Proper determination of SR is contingent on the determination of the proper peaks and troughs in the waveform. In one implementation, the peak is detected by first estimating an initial, candidate SR based on the largest pseudo-slope

$$\frac{d {y}^{2}}{d x} ,$$

where dy is the change in pulse amplitude and dx is change in time, through various run distances dx from 60-miliseconds through the estimated period (P0), which his derived from the estimated F0. A pseudo-slope array is then generated, with the pseudo-slope array representing

$$\frac{d {y}^{2}}{d x}$$

with a fixed run (dx) based on the candidate SR. The pseudo-slope array is used to estimate the center of the SR feature of the PPG. It is this feature (systolic slope), which is used, rather and a single point to find the peaks and troughs.

Peaks and troughs are then located by reviewing the PPG waveform with a center index defined as the maxima of the pseudo-slope array. First, the first local maxima of

$$\frac{d {y}^{2}}{d x}$$

is located, corresponding to the first systolic slope feature. Then, the trough is identified by maximizing the argument

$$\frac{d {y}^{2}}{d x} ,$$

indexing dx from the center index. Once a minima is found and confirmed, the corresponding maxima is sought. The pair of minima and maxima corresponds to the trough and peak, respectively, and are added to a peak array. The next center index is then incremented by one period. Then that center index is adjusted to correspond with the maxima slope in the slope array within ±P0/2 (within one half of a period). The process is repeated throughout the entire PPG waveform to generate a peak array identifying the pair of troughs and peaks for each period in the PPG waveform.

In summary, peaks may be identified according to the following criteria:
occurs in pairs
occur nominally at a distance of P0
surround the steepest, longest rise portions of the signal (the Systolic Rise period 525, M2=dy²/dx)
troughs are identified by: maxargₓ(dy²/dx) from centers of M2
troughs are not on edges of signal array
peaks occur after troughs
peaks are>surround two points
distance (d) between trough & peak is: 15% P0≦d≦50% P0

### Comparison Calculations

Further diagnostic accuracy may be achieved with subsequent limb or digit measurements and variable calculations. For example, comparisons of the same calculation taken from the toe to a finger, or from a left toe to right toe, may yield relative variables which are clinically indicative of disease. As an example, the Circulation Index (CI) of the toe may be compared to the hand in various fashions such as a ratio (e.g., CIₜₒₑ/CI_{finger}) or difference (e.g., CIₜₒₑ−CI_{finger}). Further, a toe-to-finger variable may use various bases (e.g., divisor or subtrahend) such as: average, “best”, maximum, or minimum (e.g., CI_{left-toe}/CI_{AVG(fingers)} or CI_{left-toe}/CI_{MAX(fingers)}). Additionally, a given toe may be compared to the contralateral toe or the average of the toes (e.g., CI_{left-toe}/CI_{AVG(toes)}). It should be recognized that each variant described here may be applied to each per-limb variable (e.g. CI, PI, Harmonic Slope, etc.).

The “best” basis is simply the hand, position of an organ, or limb in general, which is selected based on a particular quality. For example, the best hand might be selected based on the maximum SNR or CI among the two hands. Therefore, that hand is used as the basis for each comparison calculation.

Therefore, several variables may be used to estimate the probability of flow obstruction of a given limb by using the absolute limb variables (e.g., CI_{left-toe}) in addition to the relative limb variables (e.g., CI_{left-toe}/CI_{AVG(fingers)}).

### Predictive Model

A prediction model for flow obstruction may be generated using the absolute and relative variables. One implementation uses a logistic function of the following form:

$$P ( Dx ) = \frac{1}{1 + {e}^{( {C}_{0} + {C}_{1} {v}_{1} + {C}_{2} {v}_{2} + … + {C}_{n} {v}_{n} )}} , \qquad ( 22 )$$

where P(Dx) is the probability of flow obstruction, v₁, v₂, . . . vₙ are the absolute and relative variables based on the calculations as discussed above, and C₀, C₁, C₂, . . . Cₙ are the coefficients corresponding to each variable.

In order to determine the predictive model specific for the type or model of sensor, sensor data is taken, for example, from each limb from a sample population of test subjects with known diagnoses. Using the techniques described above, the signals from each limb are processed to calculate per-limb variables and comparative variables characterizing the sensor data as described above. Using a logistic regression model performed on aggregate data from normal and abnormal limb measurements, the coefficients for each variable are determined.

It should be noted that the coefficients for some variables may be zero, meaning the variable is not statistically significant and may be omitted. Equation 22 yields values ranging from 0.0 to 1.0 with values below a certain threshold (e.g., 0.5) suggesting the limb is flow obstructed.

The aforementioned threshold may be determined based on a sensitivity analysis, considering the sensitivity, specificity, and accuracy. In one embodiment, the sensitivity may be favored over specificity. In another embodiment, the threshold may be set based on maximizing the accuracy.

It is understood that the value of the coefficients and threshold are readily determinable by one of skill in the art using the above described methodology.

As a specific example, one embodiment uses the following equation for the prediction of flow obstruction:

$$P ( Dx ) = \frac{1}{1 + {e}^{( {C}_{0} + {C}_{1} {v}_{1} + {C}_{2} {v}_{2} + {C}_{3} {v}_{3} + {C}_{4} {v}_{4} + {C}_{5} {v}_{5} + {C}_{6} {v}_{6} )}} , \qquad ( 23 )$$

where the variables used and the corresponding coefficients include the following values and 95% confidence interval:

| (24) | | |
|---|---|---|
| Coefficient | Variable Name | Value (95% CI) |

| | | | |
|---|---|---|---|
| C₀ | Intercept | 18.05 | (±2.06) |
| C₁ | CI (foot/handsMAX) | −36.35 | (±2.41) |
| C₂ | Harm.Slope(foot/handsMAX) | 4.09 | (±0.29) |
| C₃ | Harm.Int | −7.81 | (±0.65) |
| C₄ | Harm.Int(foot-handsMAX) | 5.82 | (±0.70) |
| C₅ | Spec.Sig(foot/handsMAX) | −2.71 | (±0.57) |
| C₆ | Syst.Rise | 43.71 | (±1.90), |

In one embodiment, the sensor may include memory, which is configured so that the relevant coefficients are loaded into memory and downloaded to the host computer. The relevant coefficients and variables calculated for the predictive model may be different depending on the type or model of the sensor connected to the host computer. In another embodiment, the relevant coefficients are loaded into memory and downloaded to the host computer through a memory card inserted into the host computer. In yet another embodiment, the relevant coefficients are loaded into memory and downloaded to the host computer through a USB drive. In another embodiment, coefficients are loaded to the host computer from a remote server.

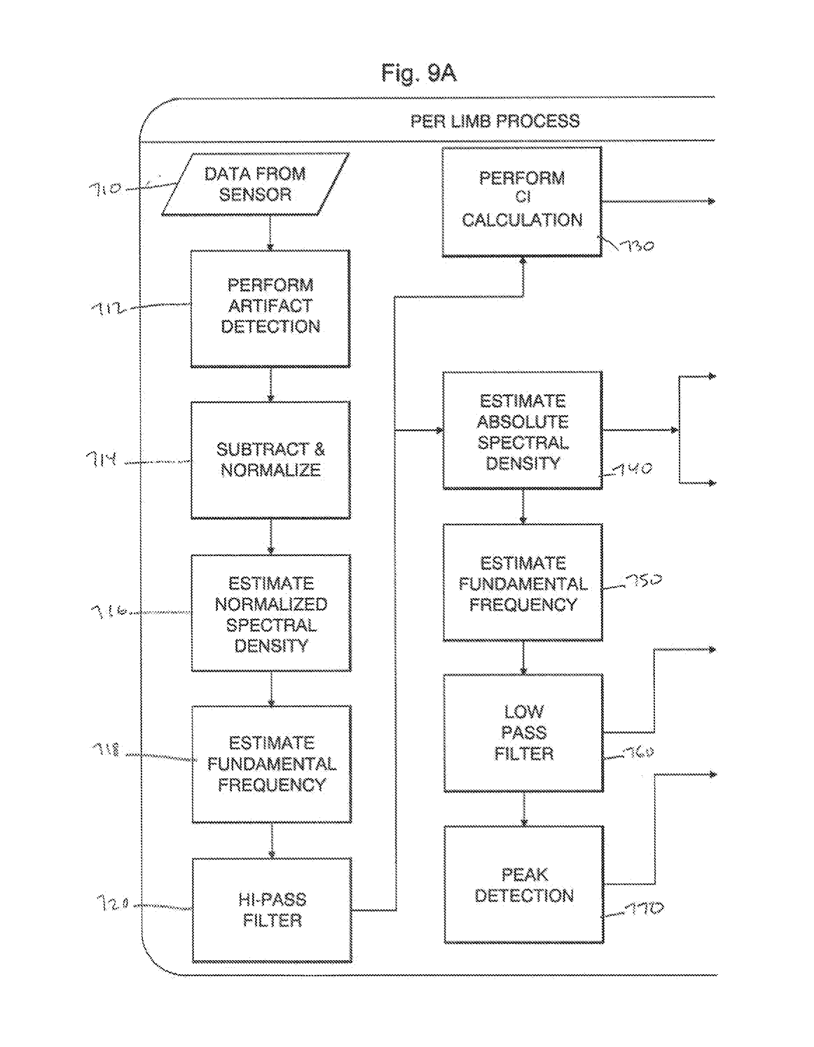

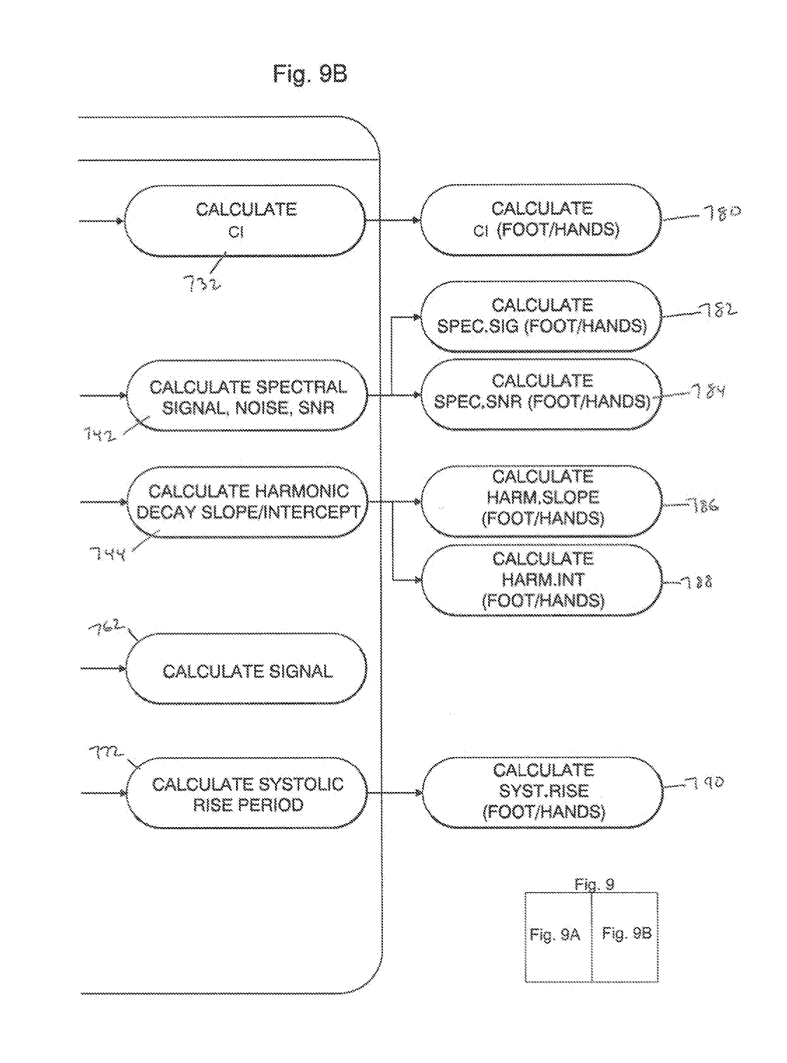

FIGS. 9A and 9B show a process flow diagram illustrating the operational steps of the multi-faceted algorithm in one example implementation for a patient with four functional limbs, namely a right arm, left arm, right foot, and left foot. In this example, a PPG sensor connected to a host computer is attached to each limb in turn to allow the PPG sensor to obtain data.

For each limb, the PPG sensor acquires a data signal as in step 710. The host computer, or alternatively the sensor itself, then identifies any signal artifacts as shown in step 712. Signals are then subtracted and normalized as shown in step 714. As shown in step 716, a normalized spectral density is estimated for the acquired sensor signal, and fundamental frequency and harmonics are identified in step 718. The signal is processed through a high-pass filter in step 720 to remove low frequency noise components.

After the low frequency noise has been removed, the signal may be processed in a number of different ways to calculate different variables. In one path, the Circulation Index (CI) is calculated in step 730 to yield a CI for the particular limb being evaluated in step 732.

Although not explicitly shown in FIGS. 9A and 9B, it will be understood that the PI may be calculated for each in place of or in addition to the calculation of the CI.

The signal may also be processed to estimate the absolute spectral density of the PPG waveform as in step 740. From this step, the spectral signal, spectral noise, and spectral signal to noise ratios can be calculated as shown in step 742. From the same information, the harmonic slope and harmonic intercept may also be calculated as shown in step 744.

From step 740, the fundamental frequencies of the spectral density can be determined in step 750. The signal can then be passed through a low pass filter in step 760 from which the AC amplitude of the signals can be calculated in step 762. The low pass filter step serves to attenuate higher frequency components of the signal to facilitate reliable peak and trough detection in step 770.

Once peak and troughs have been detected in step 770, the systolic rise period can be calculated in step 772 as previously described.

This process is repeated for each of the four limbs on a patient. Once data from all four limbs have been collected, the host computer calculates the comparative variables such as those shown in steps 780, 782, 784, 786, 788, and 790.

Having calculated the value of the comparative variables, the host computer then calculates a predictive diagnosis using the calculated comparative variables and a set of predetermined coefficients. The predictive diagnosis may be displayed on an indicator, such as a monitor attached to or integral to the host computer.

Those of skill in the art will appreciate that certain illustrated functional blocks can be omitted, reordered, combined, or separated, within the spirit and cope of the invention. Similarly, those of skill will appreciate that certain illustrated software steps can be omitted, reordered, combined, or separated, also within the spirit and scope of the invention. All such suitable topologically and logically suitable alternatives to the process flow diagramed in FIGS. 9A and 9B are contemplated as being within the spirit and scope of the invention. Further, while illustrated as being implemented via software, such steps may be suitably implemented via hardware and/or firmware.

Those of skill in the art will also appreciate that while the above steps are described in specific terms for a PPG sensor, different sensor technologies may be used. Additionally, while the examples provided above indicate the use of hands and feet, or fingers and toes, other bodily organs maybe interrogated in the same manner.

### Experiment

In one experiment using a PPG sensor, the objective of the experiment was to develop an algorithm predictive of peripheral arterial flow obstruction (FO) using data from a PPG sensing device.

Raw data from a PPG sensor was collected from 70 patients. Fifty-five patients presented to five vascular centers with signs or symptoms suggestive of peripheral arterial disease. Each patient was tested with a PPG device bilaterally on each index finger & each second toe and subsequently assessed with a proven imaging modality (e.g., Duplex ultrasound, angiography, etc.). In addition, fifteen normal control subjects were measured in the same fashion, but without the additional imaging modality.

The raw data files from the PPG sensor were then analyzed with a software application that performed calculations of several different variables including: the Circulation Index (CI), Systolic Rise (SR), Harmonic Slope (HS), Harmonic Intercept (HI), Spectral Signal (SS), Spectral Noise (SN), Spectral Signal-to-Noise Ratio (SNR). In addition, relative variables were calculated for the toes as compared to the fingers. Limbs with FO were assigned a diagnosis value of 1; while those absent FO were assigned a 0. This data was then evaluated with XLStat (version 2012.5.02) using a logistic regression analysis in a Monte Carlo simulation. That is, 107 limbs were selected at random, a logistic model created, and then validated against the other 30 limbs.

From 70 subjects and 137 limbs, 74 limbs had flow obstruction and 63 were absent of flow obstruction. The variables of significance included those listed in Equation 24: CI (foot/handsMAX), Harm.Slope(foot/handsMAX), Harm.Int, Harm.Int(foot-handsMAX), Spec.Sig(foot/handsMAX), and Syst.Rise. The median accuracy in the prediction of FO was 89.7%.

It will be understood that the present invention is not limited to the method or detail of construction, fabrication, material, application or use described an illustrated herein. Indeed, any suitable variation of fabrication, use, or application is contemplated as an alternative embodiment, and thus is within the spirit and scope of the invention.

It is further intended that any other embodiments of the present invention that result from any changes in application or method of use or operation, configuration, method of manufacture, shape, size, or material, which are not specified within the detailed written description or illustrations contained herein yet would be understood by one skilled in the art, are within the scope of the present invention.

Finally, those of skill in the art will appreciate that the invented method, system and apparatus described and illustrated herein may be implemented in software, firmware or hardware, or any suitable combination thereof. Preferably, the method and apparatus are implemented in a combination of the three, for purposes of low cost and flexibility. Thus, those of skill in the art will appreciate that embodiments of the methods and system of the invention may be implemented by a computer or microprocessor process in which instructions are executed, the instructions being stored for execution on a computer-readable medium and being executed by any suitable instruction processor.

Accordingly, while the present invention has been shown and described with reference to the foregoing embodiments of the invented apparatus, it will be apparent to those skilled in the art that other changes in form and detail may be made therein without departing from the spirit and scope of the invention as defined in the appended claims.

1. A peripheral arterial flow obstruction detection system comprising:
a housing contoured to receive a portion of a peripheral limb;
a plethysmography signal sensor in the housing and generating a pulse waveform output based on detected arterial flow;
a host computer operably coupled with said sensor;
the host computer configured to calculate values based on the pulse waveform, including at least two calculations selected from the group consisting of a time-domain feature calculation and a frequency-domain feature calculation;
where n has a value equal to the sum of the number of time-domain feature calculations and frequency-domain feature calculations performed by the host computer,
the host computer being further configured to calculate a predictive diagnosis using the equation
$$P ( Dx ) = \frac{1}{1 + {e}^{( {C}_{0} + {C}_{1} {v}_{1} + {C}_{2} {v}_{2} + … + {C}_{n} {v}_{n} )}} ,$$
wherein
P(Dx) is the probability of flow obstruction;
v₁ through vₙ comprise at least two calculations selected from the group comprising time-domain feature calculations and frequency-domain feature calculations;
coefficients c₀ through cₙ are predetermined coefficients; and
an indicator configured to display the calculated predictive diagnosis.

2. The system of claim 1, wherein the sensor comprises a photodiode.

3. The system of claim 1, wherein the sensor comprises a charge coupled device (CCD).

4. The system of claim 1, wherein the physiological signal comprises infrared light.

5. The system of claim 1, wherein the coefficients c₀ through c₆ are determined through logistic regression.

6. The system of claim 1, wherein the plethysmography signal sensor is a photoplethysmography sensor.

7. The system of claim 1, wherein the plethysmography signal sensor is a volume plethysmography sensor.

8. A circulation obstruction detection system comprising:
a housing;
a sensor in the housing positioned to receive a physiological signal indicative of circulation;
a host computer operably coupled with said sensor to receive the physiological signal and configured to analyze the physiological signal and compute at least two calculations selected from the group consisting of a) a time-domain feature calculation, and b) a frequency-domain feature calculation;
the host computer being further configured to calculate a predictive diagnosis using said at least two calculations, in a logistic function that predicts probability of circulation obstruction; and
an indicator configured to display the calculated predictive diagnosis.

9. The system of claim 8, wherein the sensor comprises a photodiode.

10. The system of claim 8, wherein the sensor comprises a charge coupled device (CCD).

11. The system of claim 8, wherein the sensor comprises a pressure cuff configured with a pressure transducer.

12. The system of claim 8, wherein the sensor comprises a strain gauge.

13. The system of claim 8, wherein the physiological signal comprises infrared light.

14. The system of claim 8, wherein the physiological signal comprises organ volume.

15. The system of claim 8, wherein the physiological signal comprises dermal temperature.

16. The system of claim 8, wherein the physiological signal comprises dermal tension.

17. The system of claim 8, wherein the physiological signal comprises blood velocity.

18. A peripheral arterial flow obstruction detection system comprising:
a housing contoured to receive a portion of a peripheral limb, the housing further comprising a sensor capable of detecting a photoplethysmographic signal from the portion of a peripheral limb, the sensor generating a pulse waveform;
a host computer operably coupled with said sensor, the host computer configured to obtain values based on the pulse waveform, including the circulation index, harmonic slope, harmonic intercept, spectral signal, and systolic rise;
the host computer being further configured to calculate a predictive diagnosis using the equation
$$P ( Dx ) = \frac{1}{1 + {e}^{( {C}_{0} + {C}_{1} {v}_{1} + {C}_{2} {v}_{2} + {C}_{3} {v}_{3} + {C}_{4} {v}_{4} + {C}_{5} {v}_{5} + {C}_{6} {v}_{6} )}} ,$$
wherein
P(Dx) is the probability of flow obstruction;
v₁ is the circulation index (foot/handsMAX);
v₂ is the harmonic slope (foot/handsMAX);
v₃ is the harmonic intercept;
v₄ is the harmonic intercept (foot-handsMAX);
v₅ is the spectral signal (foot/handsMAX);
and v₆ is the systolic rise;
coefficients c₀ through c₆ are predetermined coefficients; and
an indicator configured to display the calculated predictive diagnosis.

19. The system of claim 18, wherein:
c₀ has a value ranging from 15.99 to 20.11;
c₁ has a value ranging from −33.94 to −38.76;
c₂ has a value ranging from 3.80 to 4.38;
c₃ has a value ranging from −7.16 to −8.46;
c₄ has a value ranging from 5.12 to 6.52;
c₅ has a value ranging from −2.14 to 3.28; and
c₆ has a value ranging from 41.81 to 45.61.

20. The system of claim 19, wherein:
c₀ has a value of 18.05;
c₁ has a value of −36.35;
c₂ has a value of 4.09;
c₃ has a value of −7.81;
c₄ has a value of 5.82;
c₅ has a value of 2.71; and
c₆ has a value of 43.71.
